(19) United States
(12) Patent Application Publication
(10) Pub. No.: US 2026/0021082 A1
(43) Pub. Date: Jan. 22, 2026

(54) EZH2 INHIBITION THERAPIES FOR THE TREATMENT OF AT-RICH INTERACTIVE DOMAIN-CONTAINING PROTEIN 1A (ARID1A) MUTATED CANCERS

(71) Applicant: Constellation Pharmaceuticals, Inc., Boston, MA (US)

(72) Inventors: Elizabeth Adams Whicher, Boston, MA (US); Ziyang Yu, Boston, MA (US); Kaiming Sun, Belmont, MA (US); Patricia Joyce Keller, Shorewood, MN (US); Patrick Trojer, Reading, MA (US)

(21) Appl. No.: 18/994,995

(22) PCT Filed: Jul. 14, 2023
(86) PCT No.: PCT/US2023/027761

§ 371 Date: Jan. 15, 2025

Related U.S. Application Data
(60) Provisional application No. 63/389,436, filed on Jul. 15, 2022.
(60) Provisional application No. 63/413,323, filed on Oct. 5, 2022.
(60) Provisional application No. 63/455,024, filed on Mar. 28, 2023.

(51) Int. Cl. A61K 31/443; A61P 35/00

(52) U.S. Cl. CPC A61K 31/443; A61P 35/00

(57) ABSTRACT

Provided herein are methods of treating cancers having at least one ARID 1 A mutation with (R)-7-chloro-2-((1r,4R)-4-(3-methoxyazetidin-1-yl)cyclohexyl)-2,4-dimethyl-N-((6-methyl-4-(methylthio)-2-oxo-1,2-dihy-dropyridin-3-yl)methyl)benzo[d][1,3]dioxole-5-carboxamide, or a pharmaceutically acceptable salt thereof.

### RELATED APPLICATIONS

This application claims priority to U.S. provisional application No. 63/389,436, filed Jul. 15, 2022, U.S. provisional application No. 63/413,323, filed Oct. 5, 2022, and U.S. provisional application No. 63/455,024, filed Mar. 28, 2023, the entire contents of each of which are incorporated herein by reference.

### BACKGROUND

Mutations of the ARID1A gene, which encodes the basic directional subunit of SWI/SNF chromatin remodeling complexes, have manifested in several groups of cancers including subtypes of ovarian, endometrial, and uterine cancers. ARID1A is a tumor suppressor, where its loss leads to increased cell proliferation, migration, and invasion, as well as reduced cell apoptosis and chemosensitivity. ARID1A is mutated in 25% of muscle-invasive bladder cancer and a high frequency of ARID1A mutations has been reported in a number of indications, including ovarian clear cell carcinoma (46-57%), endometrial cancers (30-40%), and gastric cancer (20%).

Despite efforts to combine multiple cancer immunotherapeutic agents and ongoing clinical exploration of cancer immunotherapy combinations with other therapies, ARID1A mutated cancers, particularly advanced urothelial carcinoma, a subtype of bladder cancer, remains largely incurable with only a minority of patients responding in second or later lines of treatment with limited survival benefits. New approaches to target cancers harboring one or more ARID1A mutations are therefore needed.

### SUMMARY

It has now been found that cancers with at least one ARID1A mutation have increased phenotypic sensitivity to the EZH2 inhibitor (R)-7-chloro-2-((1r,4R)-4-(3-methoxyazetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide, herein referred to as Compound 1. See e.g., FIG. 1, FIG. 19 and Table 1, where growth inhibitory effects were significantly enriched (p=3.7e−6, Chi-square test) in a panel of 21 bladder cancer cells lines following treatment with Compound 1 in cell lines carrying at least one ARID1A loss of function (LOF) allele, with 83% (5 out of the 6) sensitive cell lines (GI50 of 3-37 nM after 18 days) harboring a truncation mutation (frameshift or nonsense). By contrast, only 6% (1 out of 15) of unresponsive cell lines with an 18-day GI50>5 μM, had an ARID1A LOF allele. Additionally, treatment with Compound 1 significantly inhibited tumor growth in patient-derived xenograft (PDX) models of ARID1A LOF endometrial cancers (FIG. 7) and in an ARID1A mutant TOV21G cell line-derived xenograft (CDX) models of ovarian clear cell carcinoma (OCCC) (FIG. 8). Surprisingly, however, colon cancers, lung cancers, and non-clear cell ovarian cancers having ARID1A mutations did not show increased sensitivity to treatment as a whole. See e.g., FIG. 12, where no apparent correlation between mutation status and responsiveness was observed. Clinical efficacy in subjects with cancers harboring ARID1A mutations was also observed (FIG. 13, FIG. 14). Tumor mutational burden was found to be low for most patients with OCCC or EC. Therefore, in one aspect, provided are methods of using Compound 1 to treat ARID1A mutated cancers with low tumor mutational burden. In one aspect the tumor mutational burden is below 10 mut/Mb.

It has also been found that Compound 1 shows a higher response rate in some ARID1A mutated PDX cancer models compared to the response rate in corresponding ARID1A wild-type cancer indications (Table 6). Therefore, in one aspect, some ARID1A mutated cancers show a higher response rate than corresponding ARID1A wild-type cancers.

In one aspect, therefore, provided are methods of using Compound 1 to treat a cancer having at least one ARID1A mutation. Also provided are uses of Compound 1 for the manufacture of medicaments for treating said cancers.

It has also been found that Compound 1 restores ARID1A expression in ARID1A mutant bladder cancer cells. For example, GSEA of genes upregulated after Compound 1 treatment showed enrichment in ARID1A re-expression targets. See e.g., FIG. 11. Therefore, in one aspect, provided are methods of using Compound 1 to restore ARID1A expression in cancers having at least one ARID1A allele. Also provided are uses of Compound 1 for the manufacture of medicaments for restoring ARID1A expression in cancers having at least one ARID1A allele.

### BRIEF DESCRIPTION OF THE FIGURES

FIG. 1 shows the results from an 18-day viability assay GI50 values for Compound 1 in a panel of bladder cancer cell lines. Black bar indicates cell line carrying at least one ARID1A stop-gain (denoted by an *) or frameshift (fs) allele as detailed below the chart. Green bar indicates line carrying a single missense mutation, while grey bars indicate those lines with no mutations in the coding region of ARID1A. Data represented as an average of duplicate wells±SD and are representative of duplicate independent experiments.

FIG. 2 is a summary of the mutation status of the major components of the BAF complex as well as KDM6A in the bladder cancer panel. Those noted in bold are the most frequently mutated in cancer.

FIG. 3 represents normalized global H3K27me levels in HT1197 (left) and T24 (right) cell lines following 72 hours of treatment across a dose range of Compound 1. Data represented as average of triplicate wells±SD and are representative of quadruplicate independent experiments.

FIG. 4 shows cell viability dose response curves in HT1197 (left) and T24 (right) cell lines over 18 days of treatment. Data represented as an average of duplicate wells±SD and are representative of duplicate independent experiments.

FIG. 5 shows tumor growth inhibition from Compound 1 or vehicle treatment in HT1376 bladder cancer xenografts. Data represented as mean tumor volume+SEM, with n=5 mice per group for all groups except 150 mg/kg, which had n=3. P-values calculated using 2-way ANOVA up to day 30, ns=p>0.05, *p<0.05.

FIG. 6 shows tumor growth inhibition in an ARID1A mutant PDX model of bladder cancer (BL9209) treated with Compound 1, 75 mg/kg PO, QD. Data represented as mean tumor volume+SEM, with n-3 mice per group. TGI calculated using tumor volumes at day 28, *p<0.05 using 2-way ANOVA through day 28, when vehicle reached endpoint.

FIG. 7 shows tumor growth inhibition in an ARID1A mutant PDX model of endometrial cancer (UT5319) treated with Compound 1. Mice were initially treated with 75 mg/kg PO QD, then dose was reduced to 50 mg/kg PO QD after d23. Data represented as mean tumor volume +SEM, with n=3 mice per group. TGI calculated using tumor volumes at day 21, ***p<0.0001 using 2-way ANOVA through day 21, when vehicle reached endpoint.

FIG. 8 shows tumor growth inhibition from Compound 1 or vehicle treatment in TOV21G OCCC xenografts. Data represented as mean tumor volume+SEM, with n=6 mice in vehicle arm, n=4 in Compound 1 arm. P-values calculated using 2-way ANOVA up to day 42, **p<0.01.

FIG. 9 shows a western blot of whole cell lysates from HT1376 cells transduced with the empty TET-ON vector or TET-ON-ARID1A, with or without 50ng/ml Dox treatment for 5 days and treated with DMSO or 250 nM Compound 1 for 4 days.

FIG. 10 shows gene set enrichment analysis (GSEA) of EZH2 target genes in HT1376 transduced with empty vector or doxycycline-inducible ARID1A and treated with 50 ng/ml doxycycline for 5 days.

FIG. 11 is the GSEA of ARID1A-induced genes (defined in Sup FIG. 3F) in HT1376 transduced with doxycycline-inducible ARID1A vector and treated with 50 ng/ml doxycycline for 5 days and either DMSO or 250 nM Compound 1 for 4 days.

FIG. 12 shows the responses from treatment with Compound 1 in ARID1A mutated PDX or PD organoid type models (as indicated) of colon cancer, lung cancer, gastric cancer, and non-clear cell ovarian cancer.

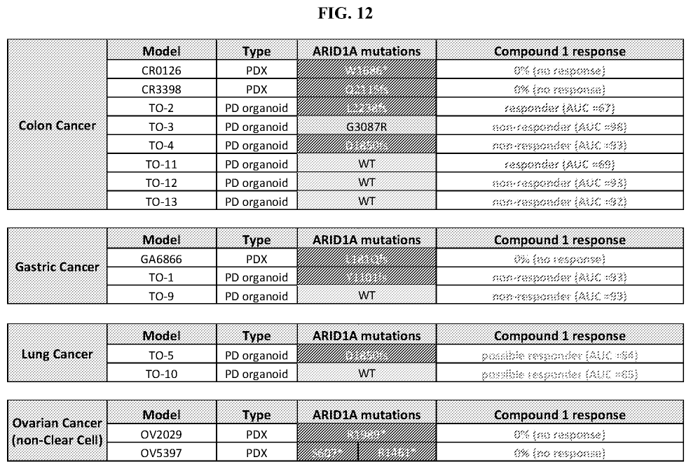

FIG. 13 shows the treatment duration of Compound 1 in subjects with ovarian clear cell carcinoma harboring ARID1A mutations.

FIG. 14 shows the treatment duration of Compound 1 in subjects with endometrial carcinoma harboring ARID1A mutations.

FIG. 15 shows the tumor volumes in an ARID1A mutant HT1376 xenograft efficacy study after treatment with various EZH2 and EED inhibitors. Data represented as mean+SEM, with n=6 mice for per group for all arms except Compound 1, which had n=12. TGI values noted were calculated for all arms using day 27 tumor volumes, relative to vehicle. P-values calculated using 2-way ANOVA, *p<0.05, **p<0.01, ***p<0.0001 using 2-way ANOVA up to day 27 (for TGIs) or through day 55 (for p-values on the graph).

FIG. 16 shows H3K27me3 levels from tumor samples collected at day 15 from study depicted in FIG. 15. Data represented as mean+SD, n=3 tumors per group. P-values calculated using unpaired Student's t-test, **p<0.01, ***p<0.0001.

FIG. 17 shows a Scatter plot showing relationship between relative H3K27me3 levels and tumor size. The relative H3K27me3/H3 ratio from tumor samples collected at day 15 of each group from FIG. 15 was normalized to that of vehicle group. The tumor volume of each group was measured at day 27. P-value was calculated by Pearson correlation coefficient.

FIG. 18 shows a Bar plot of expression changes of EZH2 target genes (as defined in Table 5) at day 15 relative to vehicle for the tumors from FIG. 15. Data represented as mean Log2 fold change±95% confidence interval.

FIG. 19 shows the results from an 21-day viability assay GI₅₀ values for Compound 1 in a panel of ovarian clear cell carcinoma and endometrioid cell lines. Black bar indicates cell line carrying at least one ARID1A stop-gain (denoted by an *) or frameshift (fs) allele, as detailed below the chart. Grey bars indicate those lines with the wildtype (no mutations) coding region of ARID1A. Data represented as an average of triplicate wells in two independent experiments±SD.

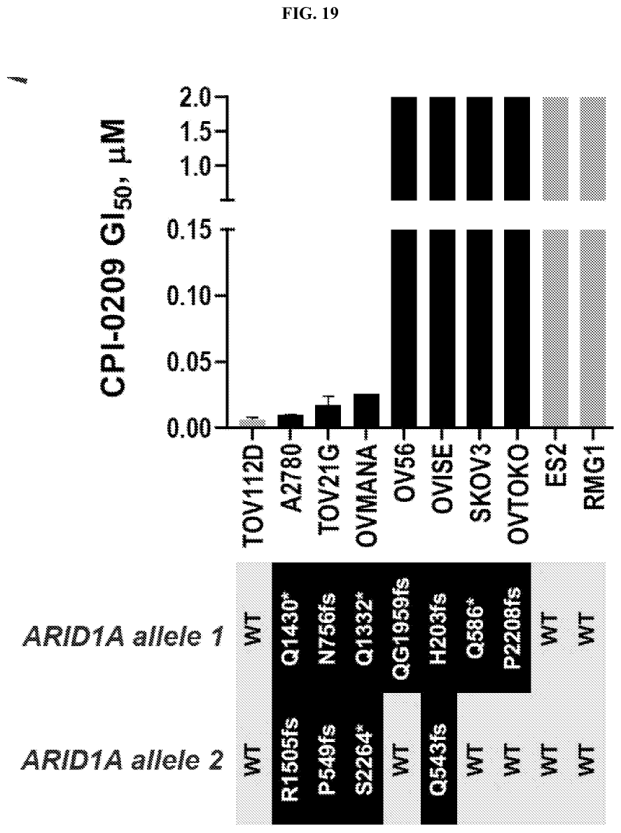

FIG. 20 shows tumor growth inhibition in an ARID1A mutant PDX model of endometrial cancer (UT5326) after treatment with Compound 1. Mice were treated with 75 mg/kg PO QD. Data represented as mean tumor volume+SEM, with n=5 mice per group. TGI calculated using tumor volumes at day 49, *p<0.05 using 2-way ANOVA through day 49, when study reached endpoint.

FIG. 21 shows tumor growth inhibition in an ARID1A mutant PDX model of endometrial cancer (CTG-1280) after treatment with Compound 1. Mice were treated with 75 mg/kg PO QD. Data represented as mean tumor volume+SEM, with n=5 mice per group. TGI calculated using tumor volumes at day 27, *p<0.05 using 2-way ANOVA through day 27, when vehicle reached endpoint.

FIG. 22 shows tumor growth inhibition in an ARID1A mutant PDX model of endometrial cancer (UT5320) after treatment with Compound 1. Mice were initially treated with 75 mg/kg PO QD, then dose was reduced to 50 mg/kg PO QD from day 12 to day 21, after which 75 mg/kg dosing was resumed. Data represented as mean tumor volume+SEM, with n=5 mice per group. TGI calculated using tumor volumes at day 17, *p<0.05 using 2-way ANOVA through day 17, when vehicle reached endpoint.

FIG. 23 shows tumor growth inhibition from Compound 1 or vehicle treatment in ARID1A mutant HT1376 bladder cancer xenografts treated 35 or 50 mg/kg PO QD for 48 days. Data represented as mean tumor volume or mean % change in body weight from day 0+SEM, with n=8 mice per group for vehicle and 50 mg/kg cohorts, and n=6 for 35 mg/kg cohort.

FIG. 24 shows global H3K27me3 levels in ARID1A mutant HT1376 xenograft tumors following 12 or 31 days of treatment. Data points represent measurement from individual tumors sampled at given timepoint and error bars reflect the mean+SEM. Two tumors were assayed at day 12 and five were assayed at day 31. All treatment groups were significantly lower than vehicle control at same timepoint, **p<0.01, ***p<0.0001, unpaired Student's t-test. No data for 150 mg/kg at day 31 as tumors were too small for analysis.

### DETAILED DESCRIPTION

In a first embodiment, provided are methods of treating a cancer in a subject comprising administering to the subject an effective amount of Compound 1, or a pharmaceutically acceptable salt thereof, wherein the cancer has at least one ARID1A mutation. Such cancers include, but are not limited to bladder cancer (e.g., urothelial carcinoma), endometrial cancer, ovarian cancer, ovarian clear cell carcinoma, breast cancer, gastric cancer, colon cancer, colorectal cancer, pancreatic cancer, cholangio cancer, stomach cancer, hepatocellular cancer, liver cancer, lung cancer, and melanoma. In one aspect, the cancer is selected from bladder cancer (e.g., urothelial carcinoma), endometrial cancer, and ovarian clear cell carcinoma.

Also provided as part of a first embodiment are uses of an effective amount of Compound 1, or a pharmaceutically acceptable salt thereof, for the manufacture of a medicament for treating a cancer having at least one ARID1A mutation. Such cancers include, but are not limited to bladder cancer (e.g., urothelial carcinoma), endometrial cancer, ovarian cancer, ovarian clear cell carcinoma, breast cancer, gastric cancer, colon cancer, colorectal cancer, pancreatic cancer, cholangio cancer, stomach cancer, hepatocellular cancer, liver cancer, lung cancer, and melanoma. In one aspect, the cancer is selected from bladder cancer (e.g., urothelial carcinoma), endometrial cancer, and ovarian clear cell carcinoma.

Also provided as part of a first embodiment are uses of an effective amount of Compound 1, or a pharmaceutically acceptable salt thereof, for treating a cancer having at least one ARID1A mutation. Such cancers include, but are not limited to bladder cancer (e.g., urothelial carcinoma), endometrial cancer, ovarian cancer, ovarian clear cell carcinoma, breast cancer, gastric cancer, colon cancer, colorectal cancer, pancreatic cancer, cholangio cancer, stomach cancer, hepatocellular cancer, liver cancer, lung cancer, and melanoma. In one aspect, the cancer is selected from bladder cancer (e.g., urothelial carcinoma), endometrial cancer, and ovarian clear cell carcinoma.

Also provided as part of a first embodiment, are pharmaceutical compositions comprising an effective amount of Compound 1, or a pharmaceutically acceptable salt thereof, for treating a cancer having at least one ARID1A mutation. Such cancers include, but are not limited to bladder cancer (e.g., urothelial carcinoma), endometrial cancer, ovarian cancer, ovarian clear cell carcinoma, breast cancer, gastric cancer, colon cancer, colorectal cancer, pancreatic cancer, cholangio cancer, stomach cancer, hepatocellular cancer, liver cancer, lung cancer, and melanoma. In one aspect, the cancer is selected from bladder cancer (e.g., urothelial carcinoma), endometrial cancer, and ovarian clear cell carcinoma.

Compound 1 and (R)-7-chloro-2-((1r,4R)-4-(3-methoxyazetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide are used interchangeably and each refer to the compound having the following chemical structure.

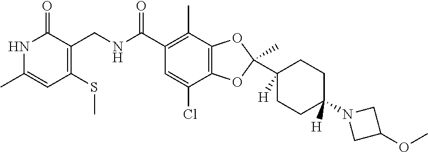

In one aspect, as part of a second embodiment, the cancer treated by the present methods is a bladder cancer. In another aspect, as part of a second embodiment, the cancer treated by the present methods is urothelial carcinoma. In another aspect, as part of a second embodiment, the cancer treated by the present methods is advanced urothelial carcinoma (e.g., urothelial carcinoma that has spread to another part of the body). In another aspect, as part of a second embodiment, the cancer treated by the present methods is endometrial cancer. In yet another aspect, as part of a second embodiment, the cancer treated by the present methods is ovarian clear cell carcinoma.

In a third embodiment, the at least one ARID1A mutation of the present methods (e.g., as in the first or second embodiment) is a loss of function (LOF) mutation. In another aspect, as part of a third embodiment, the at least one ARID1A mutation of the present methods (e.g., as in the first or second embodiment) is a truncation mutation (frameshift or nonsense). In another aspect, as part of a third embodiment, the at least one ARID1A mutation of the present methods (e.g., as in the first or second embodiment) is Q557* and the cancer is urothelial carcinoma. In another aspect, as part of a third embodiment, the at least one ARID1A mutation of the present methods (e.g., as in the first or second embodiment) is selected from G1340fs, S301fs, P302fs, P1326fs and R693, Q557* and the cancer is endometrial cancer. In another aspect, as part of a third embodiment, the at least one ARID1A mutation of the present methods (e.g., as in the first or second embodiment) is selected from Q546fs and Q723* and the cancer is ovarian clear cell carcinoma. In another aspect, as part of a third embodiment, the at least one ARID1A mutation of the present methods (e.g., as in the first or second embodiment) is selected from N1216fs and A162Rfs*238 and the cancer is endometrial cancer.

As used herein, an ARID1A LOF mutation refers to a mutation which reduces or abolishes ARID1A protein function. LOF may be due by loss of expression due to nonsense mediated decay loss of activity or due to truncation of the protein (missing critical residues or domains).

The terms “treatment,” “treat,” and “treating” refer to reversing, alleviating, delaying the onset of, or inhibiting the progress of a cancer or one or more symptoms of a disease described herein. In some embodiments, treatment may be administered after one or more signs or symptoms of a cancer have developed or have been observed (i.e., therapeutic treatment). In other embodiments, treatment may be administered in the absence of signs or symptoms of a cancer. For example, treatment may be administered to a susceptible subject prior to the onset of symptoms (i.e., prophylactic treatment) (e.g., in light of a history of symptoms and/or in light of an exposure to a pathogen). In further embodiments, treatment includes delaying the onset of at least one symptom of a cancer for a period of time. Treatment may also be continued after symptoms have resolved, for example, to delay or prevent recurrence (i.e., maintenance treatment).

The terms “subject” and “patient” may be used interchangeably, and mean a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “effective amount” or “therapeutically effective amount” refers to an amount of Compound 1, or a pharmaceutically acceptable salt thereof, that will elicit a biological or medical response of a subject e.g., a dosage of between 0.01-100 mg/kg body weight/day. In one aspect, as part of a fourth embodiment, the effective amount of Compound 1 in the present methods (e.g., in any one of the first to third embodiments) ranges from about 10 mg/kg body weight/day to about 150 mg/kg body weight/day. In another aspect, as part of a fourth embodiment, the effective amount of Compound 1 in the present methods (e.g., in any one of the first to third embodiments) ranges from about 50 mg to about 375 mg daily. In another aspect, as part of a fourth embodiment, the effective amount of Compound 1 in the present methods (e.g., in any one of the first to third embodiments) ranges from about 150 mg to about 350 mg daily. In another aspect, as part of a fourth embodiment, the effective amount of Compound 1 in the present methods (e.g., in any one of the first to third embodiments) ranges from about 175 mg to about 325 mg daily. In another aspect, as part of a fourth embodiment, the effective amount of Compound 1 in the present methods (e.g., in any one of the first to third embodiments) ranges from about 200 mg to about 300 mg daily. In another aspect, as part of a fourth embodiment, the effective amount of Compound 1 in the present methods (e.g., in any one of the first to third embodiments) ranges from about 225 mg to about 375 mg daily. In another aspect, as part of a fourth embodiment, the effective amount of Compound 1 in the present methods (e.g., in any one of the first to third embodiments) ranges from about 325 mg to about 400 mg daily. In another aspect, as part of a fourth embodiment, the effective amount of Compound 1 in the present methods (e.g., in any one of the first to third embodiments) ranges from about 350 mg to about 375 mg daily. In another aspect, as part of a fourth embodiment, the effective amount of Compound 1 in the present methods (e.g., in any one of the first to third embodiments) is about 200 mg daily. In another aspect, as part of a fourth embodiment, the effective amount of Compound 1 in the present methods (e.g., in any one of the first to third embodiments) is about 250 mg daily. In another aspect, as part of a fourth embodiment, the effective amount of Compound 1 in the present methods (e.g., in any one of the first to third embodiments) is about 300 mg daily. In another aspect, as part of a fourth embodiment, the effective amount of Compound 1 in the present methods (e.g., in any one of the first to third embodiments) is about 350 mg daily. In another aspect, as part of a fourth embodiment, the effective amount of Compound 1 in the present methods (e.g., in any one of the first to third embodiments) is about 375 mg daily. In one aspect, as part of a fourth embodiment, the effective amount of a pharmaceutically acceptable salt of Compound 1 in the present methods (e.g., in any one of the first to third embodiments) is equivalent to an amount of Compound 1 ranging from about 10 mg/kg body weight/day to about 150 mg/kg body weight/day. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 50 mg to about 375 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 325 mg to about 400 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 ranging from about 350 mg to about 375 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 of about 350 mg daily. In another aspect, as part of a fourth embodiment, the effective amount of a pharmaceutically acceptable salt of Compound 1 in the present methods (e.g., in any one of the first to third embodiments) is equivalent to an amount of Compound 1 of about 375 mg daily.

Methods of administration herein may be orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Sterile injectable forms of Compound 1 described herein may be aqueous or oleaginous suspension. These suspensions may be formulated according to techniques known in the art using suitable dispersing or wetting agents and suspending agents. In one aspect, as part of a fifth embodiment, Compound 1 in the present methods (e.g., in any one of the first to fourth embodiments) is administered orally.

Compound 1 may be present in the form of a pharmaceutically acceptable salt. For use in medicines, pharmaceutically acceptable salt refers to non-toxic “pharmaceutically acceptable salts.” Pharmaceutically acceptable salt forms include pharmaceutically acceptable acidic/anionic or basic/cationic salts where possible.

Compound 1, or a pharmaceutically acceptable salt thereof, may be formulated as part of a pharmaceutical composition comprising Compound 1, or a pharmaceutically acceptable salt thereof, together with a pharmaceutically acceptable carrier. Pharmaceutically acceptable carriers, (e.g. carriers, adjuvants or vehicles) that may be used in the compositions described herein include, but are not limited to, ion exchangers, alumina, aluminum stearate, lecithin, serum proteins (such as human serum albumin), buffer substances (such as phosphates), glycine, sorbic acid, potassium sorbate, partial glyceride mixtures of saturated vegetable fatty acids, water, salts or electrolytes (such as protamine sulfate, disodium hydrogen phosphate, potassium hydrogen phosphate, sodium chloride, zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “pharmaceutically acceptable carrier” refers to a non-toxic carrier, adjuvant, or vehicle that does not adversely affect the pharmacological activity of the compound with which it is formulated, and which is also safe for human use.

Pharmaceutically acceptable carriers, adjuvants or vehicles that may be used in the compositions of this disclosure include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (e.g., microcrystalline cellulose, hydroxypropyl methylcellulose, lactose monohydrate, sodium lauryl sulfate, and crosscarmellose sodium), polyethylene glycol, sodium carboxymethylcellulose, polyacrylates, waxes, polyethylene-polyoxypropylene-block polymers, polyethylene glycol and wool fat. In one aspect, as part of a sixth embodiment, Compound 1 in the disclosed methods (e.g., in any one of the first to fifth embodiments) is present in a crystalline form. Crystalline forms of Compound 1 are disclosed in WO 2021/016414 and are incorporated by reference herein. In another aspect, as part of a sixth embodiment, Compound 1 in the disclosed methods (e.g., in any one of the first to fifth embodiments) is of crystalline Form 1 characterized by at least three X-ray powder diffraction peaks at 20 angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by at least four X-ray powder diffraction peaks at 20 angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by at least five X-ray powder diffraction peaks at 20 angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by at least six X-ray powder diffraction peaks at 20 angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by X-ray powder diffraction peaks at 20 angles selected from 10.0°, 13.3°, 14.9°, 20.2°, 20.8°, 22.2°, and 22.5°. In another aspect, as part of a sixth embodiment, Compound 1 in the disclosed methods (e.g., in any one of the first to fifth embodiments) is of crystalline Form 1 characterized by X-ray powder diffraction peaks at 20 angles selected from 10.0°, 10.2°, 12.3°, 12.7°, 13.3°, 14.9°, 15.3°, 20.2°, 20.8°, 21.3°, 22.2°, 22.5°, and 23.8°. In another aspect, as part of a sixth embodiment, Compound 1 in the disclosed methods (e.g., in any one of the first to fifth embodiments) is of crystalline Form 1 characterized by X-ray powder diffraction peaks at 20 angles selected from 10.0°, 10.2°, 11.0°, 11.4°, 11.8°, 12.3°, 12.7°, 13.3°, 14.9°, 15.3°, 16.1°, 17.4°, 20.2°, 20.8°, 21.3°, 22.2°, 22.5°, and 23.8°. In another aspect, as part of a sixth embodiment, Compound 1 in the disclosed methods (e.g., in any one of the first to fifth embodiments) is of crystalline Form 1 characterized by x-ray powder diffraction peaks at 20 angles selected from 14.9°, 20.2°, and 20.8°. In another aspect, as part of a sixth embodiment, Compound 1 in the disclosed methods (e.g., in any one of the first to fifth embodiments) is of crystalline Form 1 characterized by x-ray powder diffraction peaks at 20 angles selected from 10.0°, 14.90, 20.2°, and 20.8°. In another aspect, as part of a sixth embodiment, Compound 1 in the disclosed methods (e.g., in any one of the first to fifth embodiments) is of crystalline Form 1 characterized by x-ray powder diffraction peaks at 20 angles selected from 10.0°, 14.9°, 20.2°, 20.8°, and 22.2°. In another aspect, as part of a sixth embodiment, Compound 1 in the disclosed methods (e.g., in any one of the first to fifth embodiments) is of crystalline Form 1 characterized by x-ray powder diffraction peaks at 20 angles selected from 10.0°, 13.3°, 14.9°, 20.2°, 20.8°, and 22.2°. In one aspect, as part of a seventh embodiment, Compound 1 in the disclosed methods (e.g., in any one of the first to fifth embodiments), or a pharmaceutically acceptable salt thereof, is present as a solid dispersion comprising amorphous (R)-N-((4-methoxy-6-methyl-2-oxo-1,2-dihydropyridin-3-yl) methyl)-2-methyl-1-(1-(1-(2,2,2-trifluoroethyl)piperidin-4-yl)ethyl)-1H-indole-3-carboxamide or a pharmaceutically acceptable salt thereof; and a pharmaceutically acceptable polymer. In some aspects, the pharmaceutically acceptable polymer is selected from polyvinylpyrrolidone (PVP), polyvinylpyrrolidone/vinyl acetate copolymer (PVP-VA), hydroxypropyl methylcellulose (HPMC), hypromellose phthalate (HPMC-P), and hypromellose acetate succinate (HPMC-AS), preferably HPMC or HPMC-AS, more preferably HPMC-AS grade M. In some aspects, the weight ratio of the pharmaceutically acceptable polymer to (R)-N-((4-methoxy-6-methyl-2-oxo-1,2-dihydropyridin-3-yl)methyl)-2-methyl-1-(1-(1-(2,2,2-trifluoroethyl)piperidin-4-yl)ethyl)-1H-indole-3-carboxamide ranges from 10:90 wt % to 90:10 wt %, from 15:85 wt % to 85:15 wt %, from 20:80 wt % to 80:20 wt %, from 25:75 wt % to 75:25 wt %, from 30:70 wt % to 70:30 wt %, from 35:65 wt % to 65:35 wt %, from 40:60 wt % to 60:40 wt %, or from 45:55 wt % to 55:45 wt %, preferably from 25:75 wt % to 75:25 wt %, from 30:70 wt % to 70:30 wt %, from 40:60 wt % to 60:40 wt %, or from 45:55 wt % to 55:45 wt %, more preferably from 20 wt % to 40 wt % or from 25 wt % to 35 wt %, or is 50%. Other aspects of the solid dispersion are described in WO 2018/136596.

In one aspect, as part of an eighth embodiment, Compound 1 in the disclosed methods (e.g., in any one of the first to seventh embodiments) or a pharmaceutically acceptable salt thereof, is administered for a period of at least about 4 days, at least about 6 days, at least about 8 days, at least about 12 days, at least about 18 days, at least about 30 days, at least about 60 days, at least about 6 months, or at least about 1 year.

### EXEMPLIFICATION

### Preparation of Compound 1

(R)-7-chloro-2-((1r,4R)-4-(3-methoxyazetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide was prepared following the procedures described in PCT/US2019/027932 and PCT/US2020/043163, each of which are incorporated by reference herein.

### Cell lines and Culture

The cell lines used were obtained from ATCC (Manassas, VA), DSMZ (Braunschweig, Germany), ECACC (Salisbury, UK or through Sigma), or JCRB (Osaka, Japan) and were grown in media recommended by the vendor (or indicated in supplementary methods table 1) and maintained at 37° C. in humidified incubators with 5% CO₂. Cell lines were maintained in T75 flasks and subcultured by releasing from plates with TrypLE solution (Thermo Fisher Scientific/Invitrogen #12604021) every 2-4 days, depending on growth kinetics of the cell line, to maintain growth at subconfluent levels.

### H3K27me3 Level Assessment in Cells and Tissues

H3K27me3 and total H3 expression levels in cells and tumor tissues were analyzed by Meso Scale Discovery (MSD) ELISA. For assays with cultured cell lines, trypsinzed cells were counted using a Countess® cell counter (Life Technologies) and plated in 100 μL of cell culture medium onto 96-well tissue culture treated plates containing Compound 1 (9 concentrations in a series of 3-fold dilutions) and incubated at 37° C. in 5% CO₂ for 24-96 hours, depending on the assay.

### In Vitro Washout Assays

HT1376 bladder cancer cells were used for washout experiments to look at prolonged effects on H3K27me3 levels and gene expression with Compound 1. Cells were plated in T75 flasks for 4 days and treated with compounds at the indicated doses or DMSO control. After 4 days, cells were washed twice with PBS and released from flasks with TrypLE solution. A portion of cells were removed and snap frozen for analysis of 4 days on-treatment by western blot and qRT-PCR. Remaining cells were counted and plated in duplicate wells with continuing compound treatment (on-treatment) or no compound treatment (washout) in 6-well plates for protein extraction and 24-well plates for RNA extraction, at a density that allowed for subconfluent growth for 1-4 additional days. Cells were harvested for both protein and RNA extraction from the on-treatment and washout wells for each compound at days 5, 6, 7 and 8 (day 5 samples are 5 days on-treatment or 4 days on-treatment+1 day washout etc.). For western blot analysis, cells were released from plates, washed with PBS and snap frozen. For qRT-PCR analysis, cells were washed and lysed directly in 24-well plates with buffer RLT+β-mercaptoethanol (Sigma #M6250), removed to snap cap tubes and frozen for later processing according to the manufacturer's instructions for the QIAGEN mini RNeasy kit.

### Western Blotting

Snap-frozen pellets of cultured cells were thawed on ice and lysed on ice for 20-30 minutes with RIPA-500 buffer (1X RIPA buffer solution (Boston BioProducts #BP-116T), 350 nM NaCl, 0.1% Benzonase, 1X cOmplete EDTA-free protease inhibitor (Roche #11873580001); NaCl adjusted to 500 nM after initial lysis). Lysates were centrifuged at 13,000 rpm for 15 minutes at 4° C. Supernatants were transferred to new microfuge tubes and protein concentration was determined by BCA method with absorbance read at A252 nm. Protein lysates were diluted if needed to the same concentration and a volume to 6× SDS Sample buffer+β-mercaptoethanol (Boston BioProducts #BP-111NR or #BP-605) to give 1X final concentration. 12-40 μg of total protein was loaded on SDS-PAGE gels (NuPAGE™ 4-12% Bis-Tris Midi Protein Gels, Invitrogen #WG1402BOX) and run with 1X NuPAGE™ MES SDS Running Buffer (Invitrogen #NP0002-02). Proteins were transferred to PVDF membrane (Immobilon-P, Millipore Sigma #IPVH00010), blocked with 1XTBST with 2% non-fat dry milk (20X Tris Buffered Saline with Tween®20, Boston BioProducts #BB-180X), and bound with antibodies for western blot analysis.

For CUT&RUN experiments, HT1376-TetON control cells and a clonal HT1376-TetON-ARID1A cell line were induced with 50 ng/ml doxycycline for 24 hours and then treated for 4 days with DMSO or 250 nM Compound 1. Two samples per condition were collected and processed as replicates for ARID1A and SMARCA4 CUT&RUN. For H3K27me3, H3K27ac, and H3K4me3, a single replicate was carried out for each CUT&RUN. Cells were fixed by adding formaldehyde to final concentration of 0.1%. Fixation was done for 1 min at room temperature. Crosslinking was stopped by adding glycine to a final concentration of 125 mM. The fixed cells were snap-frozen and shipped by Epicypher (Durham, NC) for CUT&RUN processing (see relevant sections of methods for details).

### Xenograft Tumor Studies

Cell line derived xenograft tumor experiments were performed at WuXi AppTec, Shanghi, China. Patient derived xenograft experiments were performed at Crown BioScience Inc., Taicang Jiangsu Province, China, or Champions Oncology, Rockville, MD, US. All the procedures related to animal handling, care and the treatment in the xenograft studies were performed according to the guidelines approved by the Institutional Animal Care and Use Committee (IACUC) of WuXi AppTec or Crown BioScience Inc. following the guidance of the Association for Assessment and Accreditation of Laboratory Animal Care (AAALAC). The mice were kept in individual ventilation cages at constant temperature and humidity with 3-5 animals in each cage. Mice were checked daily for any effects of tumor growth and/or treatments on normal behavior such as mobility, food and water consumption, body weight gain/loss, eye, and any other abnormal effects. Death and observed clinical signs were recorded if they occurred, and animals that were observed to be in a continuing deteriorating condition or their tumor size exceeding 3000 mm3 were euthanized before reaching a moribund state. Animal body weight was monitored regularly as an indirect measurement of toxicity. Supplemented diet was provided after cell inoculation for all groups to help with body weight maintenance. If an animal lost >15% body weight, their treatments were suspended accordingly then resumed when body weight loss returned to <10%.

### Cell Line Derived Xenograft Studies

The HT1376 and HT1197 bladder cancer cell lines and Karpas-422 lymphoma cell line were expanded in vitro under routine subculturing procedures in the medium recommended by the supplier, harvested while in the exponential growth phase, and counted for tumor inoculation. For HT1376 and Karpas-422 studies, female CB17 SCID mice at 6-8 weeks of age were used for study initiation; for the HT1197 study, female Balb/c nude mice at 6-8 weeks of age were used. Each mouse was inoculated subcutaneously in the right flank with tumor cells in 0.2 ml PBS mixed with Matrigel (BD Biosciences); 5×106 cells per injection were used for HT1376 and Karpas-422 cell lines and 1×107 cells per injection for HT1197 cells. Mice were randomized and drug treatments were started 11-15 days after inoculation when tumors reached an average of 139-160 mm3; animals were distributed so that each treatment arm had a similar starting tumor size (3-21 animals per arm, depending on experiment and sampling schedule). Mice undergoing extended dosing holidays due to body weight loss (>10 days, n=2 mice in the 150 mg/kg Compound 1 HT1376 group only), were censored from the study and not included in the data presented.

Tumor size was measured thrice weekly in two dimensions using a caliper, and the volume was expressed in mm3 using the formula: V=0.5a×b2 where a and b are the long and short diameters of the tumor, respectively. TGI was calculated for each group using the formula: TGI (%)=[1−(Ti−T0)/(Vi−V0)]×100; Ti is the average tumor volume of a treatment group on a certain day, T0 is the average tumor volume of the treatment group on the day of treatment start, Vi is the average tumor volume of the vehicle control group on the same day with Ti, and V0 is the average tumor volume of the vehicle group on the day of treatment start.

### Example 1—Phenotypic Responses Are Enriched in the Context of ARID1A LOF Mutations

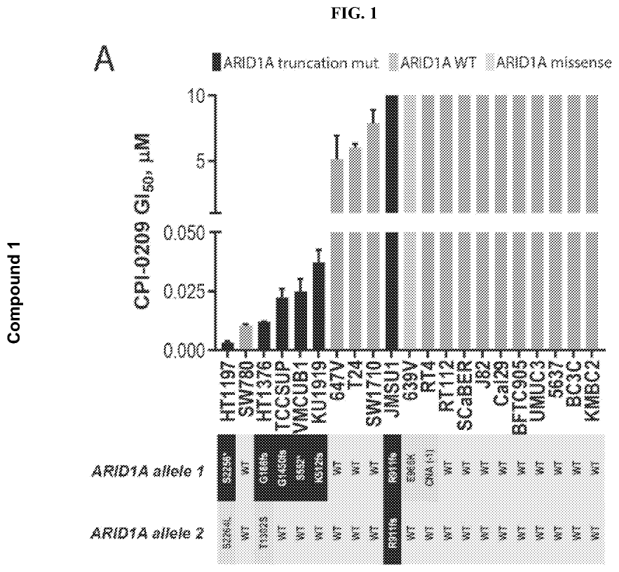

A panel of 21 bladder cancer cell lines was evaluated for their response to Compound 1. Compound 1 effectively inhibited the growth of a subset of the lines with GI₅₀ of 3-37 nM after 18 days of compound treatment. See FIG. 1 and Table 1. The growth inhibitory effects were significantly enriched (p=3.7e−6, Chi-square test) in bladder cancer lines carrying at least one ARID1A LOF allele, with 83% (5 out of the 6) sensitive cell lines harboring a truncation mutation (frameshift or nonsense) in ARID1A. By contrast, only 6% (1 out of 15) of unresponsive lines with an 18-day GI₅₀>5 μM, referred to as “resistant” from here on, have an ARID1A LOF allele. No association between Compound 1 sensitivity and baseline levels of EZH1, EZH2, H3K27me3, ARID1A or ARID1B by western blot were observed.

TABLE 1

| Cell | ARID1A | GI50 (μM) | | |
|---|---|---|---|---|
| Line | status | Day 6 | Day 12 | Day 18 |
| HT1197 | LOF mutant | >1 | >1 | 0.003 |
| SW780 | WT | 0.094 | 0.016 | 0.010 |
| HT1376 | LOF mutant | >1 | 0.047 | 0.012 |
| TCCSUP | LOF mutant | >1 | >1 | 0.022 |
| VMCUB1 | LOF mutant | >1 | >1 | 0.025 |
| KU1919 | LOF mutant | >1 | 0.168 | 0.037 |
| 647V | WT | >10 | >10 | 5.115 |
| T24 | WT | >10 | >10 | 5.992 |
| SW1710 | WT | >10 | >10 | 7.870 |
| JMSU1 | LOF mutant | >10 | >10 | >10 |
| RT112 | WT | >10 | >10 | >10 |
| RT4 | WT | >10 | >10 | >10 |
| SCaBER | WT | >10 | >10 | >10 |
| J82 | WT | >10 | >10 | >10 |
| Cal29 | WT | >10 | >10 | >10 |
| BFTC905 | WT | >10 | >10 | >10 |
| 639V | Missense | >10 | >10 | >10 |
| | mutant | | | |
| UMUC3 | WT | >10 | >10 | >10 |
| 5637 | WT | >10 | >10 | >10 |
| BC3C | WT | >10 | >10 | >10 |
| KMBC2 | WT | >10 | >10 | >10 |

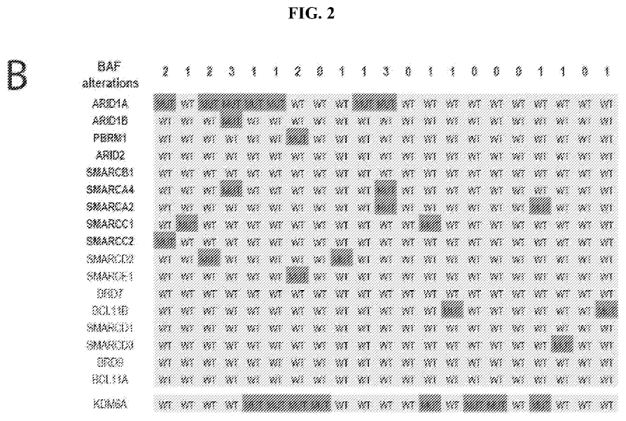

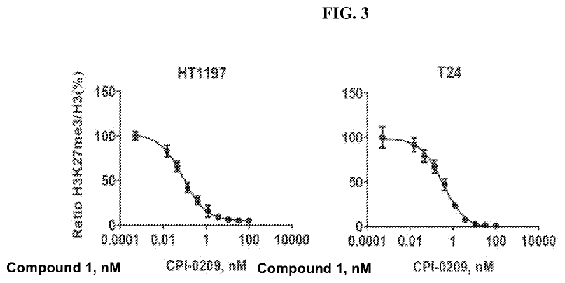

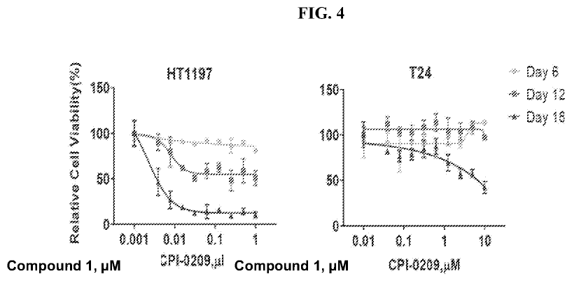

We also evaluated the mutation status of a broader set of frequently mutated BAF complex components across cancer types and found that only ARID1A genomic alterations segregate with Compound 1 response (FIG. 2), supporting the notion that ARID1A LOF mutation is a predictive biomarker for response to EZH2 inhibition. Notably, mutations in the histone demethylase gene KDM6A which, like ARID1A, is recurrently mutated in bladder cancer and is thought to potentially confer sensitivity to EZH2 inhibition, do not associate with Compound 1 response in this panel (FIG. 2). Compound 1 is equally effective at reducing H3K27me3 levels in both resistant and sensitive cell lines, with a consistent concentration-dependent reduction of H3K27me3 levels observed at 72 hours (Table 2) irrespective of phenotype or ARID1A mutation status (FIG. 3). As with EZH2 inhibitors in other cancer cell line contexts, the cell viability effects in Compound 1 sensitive bladder cancer cell lines are time-dependent. While most bladder cancer cell lines show little to no viability effects after 6 days of Compound 1 treatment, prolonged treatment for 12 and 18 days substantially increased the sensitivity of ARID1A mutant cell lines (FIG. 4).

TABLE 2

EC50 (nM) 3-day MSD

| | Repli- | Repli- | Repli- | Repli- | |
|---|---|---|---|---|---|
| | cate 1 | cate 2 | cate 3 | cate 4 | Avg |
| KU1919 | 0.533 | 0.405 | 0.414 | 0.383 | 0.434 |
| VMCUB1 | 0.117 | 0.18 | | | 0.149 |
| HT1197 | 0.077 | 0.141 | | | 0.109 |
| HT1376 | 0.148 | 0.112 | 0.127 | 0.217 | 0.151 |
| BFTC905 | 0.1 | 0.096 | | | 0.098 |
| 639V | 0.065 | 0.086 | | | 0.076 |
| T24 | 0.323 | 0.373 | 0.506 | | 0.401 |

Phenotypically responsive cell lines such as HT1376 and HT1197 showed induction of cell death on day 12, as evidenced by an increase in the subG1 population, while the cell cycle profiles of resistant cell lines such as T24 remain unchanged even after prolonged Compound 1 treatment. Taken together, these results indicate that while Compound 1 is equally effective at inhibiting PRC2's ability to maintain H3K27me3 levels, only sensitive cell lines show induction of cell death and subsequent loss of cell viability over time.

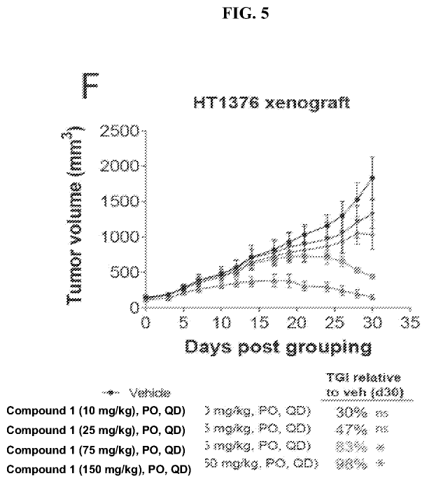

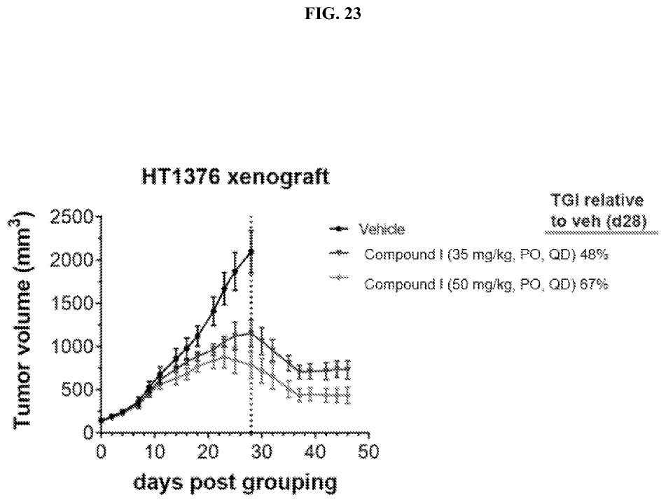

To further explore the potential dose dependence of Compound 1 on tumor growth inhibition in vivo, HT1376 xenograft studies were carried out using Compound 1 doses from 10 to 150 mg/kg QD orally once daily (PO, QD). Dose-dependent TGI was achieved with Compound 1 treatment and ranged from 30% with 10 mg/kg to 98% with 150 mg/kg by 30 days, with doses<75 mg/kg being well tolerated. All dose levels≥75 mg/kg resulted in significant reductions in tumor volume compared to vehicle (FIG. 5). Global H3K27me3 levels in HT1376 tumors were reduced in response to Compound 1 treatment in a dose-dependent manner, with at least an 80% reduction at 10 mg/kg and >90% in 25 mg/kg and higher doses at day 10 and maintained at comparable levels at day 31. A subsequent HT1376 xenograft study with an extended duration of treatment showed reproducible levels of TGI at 30 days followed by tumor volume reduction at both 35 mg/kg QD and 50 mg/kg QD dose levels at timepoints beyond 35 days (FIG. 23).

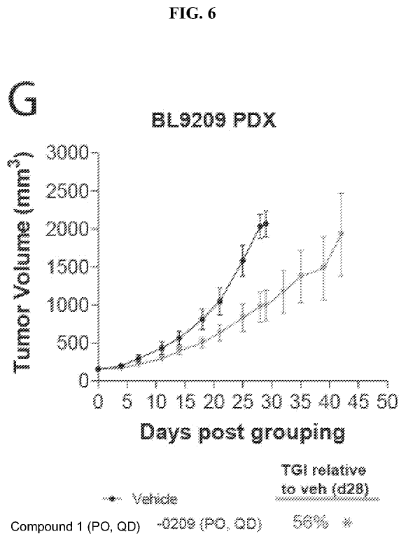

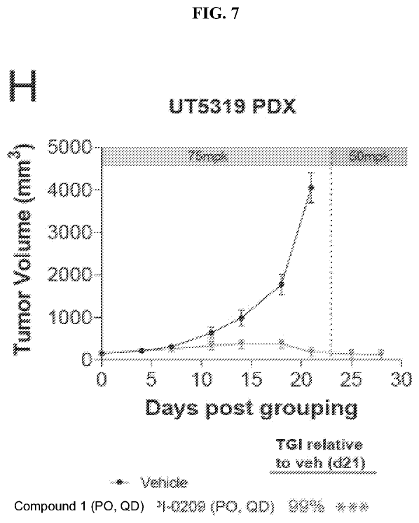

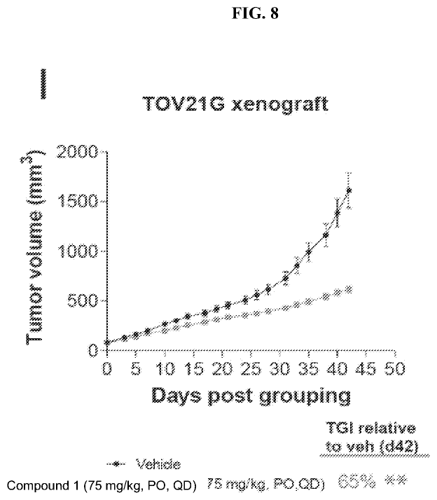

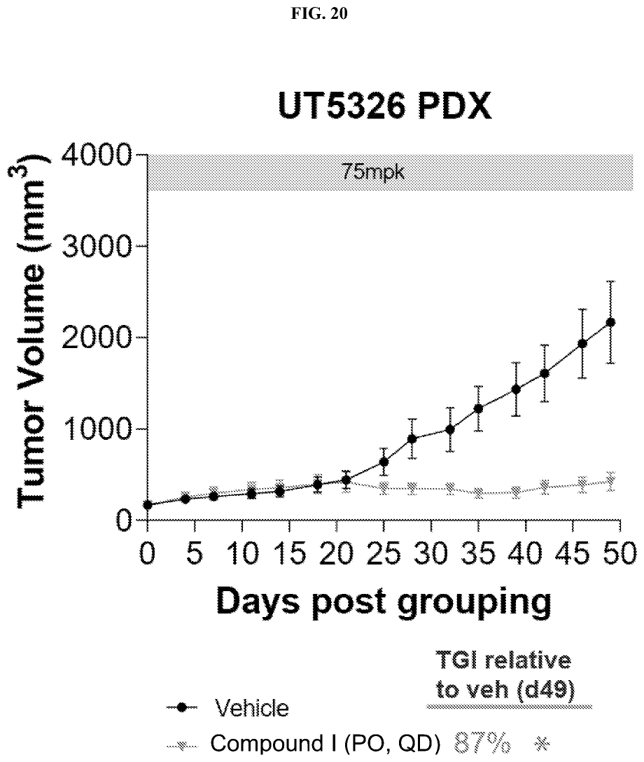

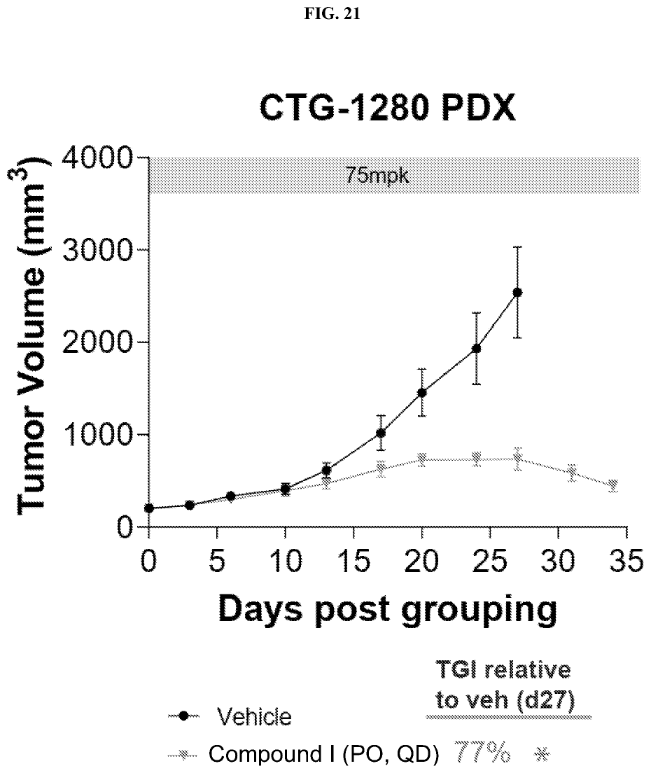

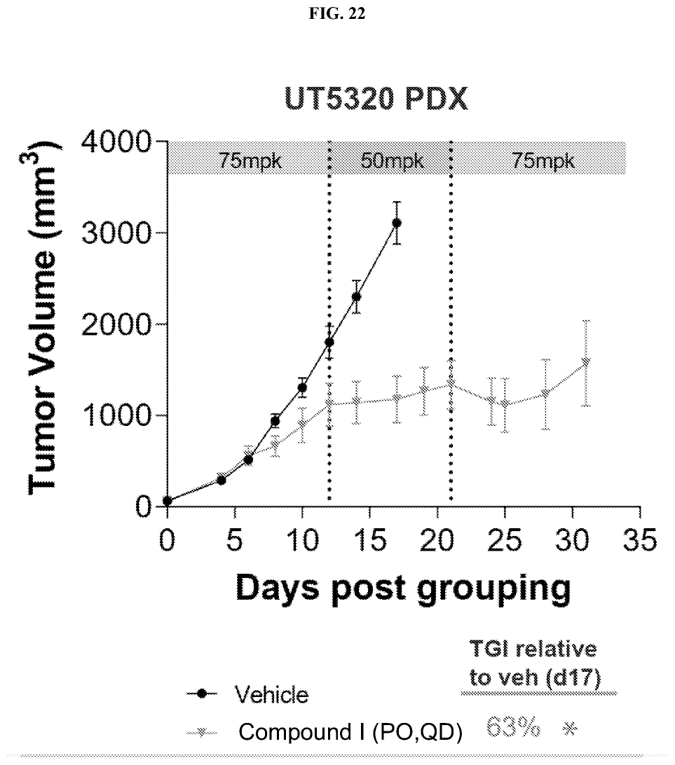

Next, we assessed in vitro and in vivo efficacy of Compound 1 in ARID1A LOF models of other solid cancers. Compound 1 also achieved significant anti-tumor activity as a single agent in patient-derived xenograft (PDX) models of ARID1A LOF bladder and endometrial cancers (FIG. 6, FIG. 7, FIG. 20, FIG. 21 and FIG. 22), as well as in the ARID1A mutant TOV21G cell line-derived xenograft (CDX) model of ovarian clear cell carcinoma (OCCC), consistent with its sensitivity to Compound 1 in vitro (FIG. 8). Together, these data demonstrate that Compound 1 is highly efficacious at well tolerated doses in ARID1A LOF solid tumor models across indications that exhibit a high frequency of ARID1A mutations. Furthermore, comparative studies of ARID1A wild-type and ARID1A mutated PDX cancer models were performed (Table 6).

TABLE 6

| | ARID1A wild-type | | ARID1A mutated | |
|---|---|---|---|---|
| Model | Responder | | Responder | |
| [Responder | (n out of | % | (n out of | % |
| TGI > 50%] | x tested) | responder | x tested) | responder |
| Breast Cancer | 3/8 | 38 | 5/6 | 83 |
| Lung Cancer | 4/8 | 50 | 3/7 | 43 |
| Gastric Cancer | 2/9 | 22 | 4/8 | 50 |
| Colorectal | 5/8 | 63 | 3/7 | 43 |
| Cancer | | | | |

### Example 2—Compound 1 Treatment Phenocopies Restoration of ARID1A Expression in ARID1A Mutant Bladder Cancer Cells

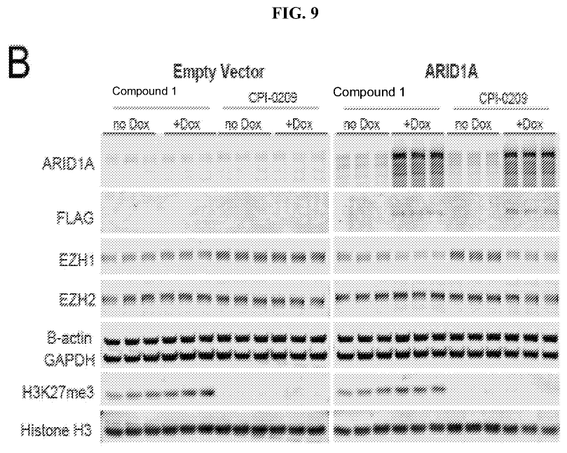

To study molecular changes underpinning the increased potential for EZH2 dependency in the context of ARID1A mutations, we expressed wildtype ARID1A in the ARID1A mutant cell model HT1376 using a doxycycline-inducible system. Significant reduction of H3K27me3 levels in HT1376 cells treated with Compound 1 were observed regardless of ectopic ARID1A expression. While EZH1 levels increased with Compound 1 treatment, no change in EZH2 was seen under any of the tested conditions (FIG. 9). Restoration of ARID1A function in HT1376 cells resulted in loss of cell viability to a similar degree as treatment with Compound 1 alone, and the combination of ARID1A re-expression and Compound 1 treatment did not result in combinatorial growth defects, suggesting that both EZH2 inhibition and ARID1A re-expression dramatically impact viability of cells that have adapted to an altered epigenetic state due to ARID1A loss. We next sought to explore changes in chromatin binding profiles and gene expression in HT1376 cells following re-expression of ARID1A and/or Compound 1 treatment. We identified three clusters of active enhancers, as determined by positivity for histone H3 lysine 27 acetylation (H3K27ac), with distinct behaviors in response to ARID1A or Compound 1. Enhancer cluster 1 was uniquely defined by low H3K27ac and high H3K27me3 levels and shows increased enhancer activity following Compound 1 treatment, as evidenced by elevated H3K27ac levels and dramatic reduction of H3K27me3 baseline levels. Enhancer clusters 2 and 3 also showed increased enhancer activity in response to Compound 1 treatment, but to lesser degree than enhancer cluster 1. Clusters 2 and 3 (to a lesser extent) were primarily defined by increase both ARID1A and SMARCA4 binding in response to ARID1A re-expression. A combinatorial increase in ARID1A and SMARCA4 occupancy was seen in enhancer cluster 1 when cells both re-expressed ARID1A and were treated with Compound 1, suggesting these enhancers are associated with gene targets that are co-regulated by PRC2 and BAF. Unsupervised clustering revealed 3 subclusters: a small subset of genes repressed by ARID1A but unchanged by Compound 1 (enhancer-proximal subcluster 1A), genes induced by Compound 1 but not ARID1A (enhancer-proximal subcluster 1B), and genes induced by either Compound 1, ARID1A, or both (enhancer-proximal subcluster 1C). Compound 1 induced genes within enhancer-proximal subcluster 1C are enriched for PRC2 targets, targets of the chimeric oncogenic transcription factor PAX3-FOXO1, and p53 targets. Enhancer-proximal subcluster 1B enriched for PRC2 targets, but no gene sets are significantly enriched in enhancer-proximal subcluster 1A.

Analysis of CUT&RUN peaks on the gene body and flanking regions identified two clusters of genes, which both shift (to different degrees) from a repressive state (high H3K27me3/low H3K27ac) to a more permissive state (low H3K27me3/high H3K27ac) upon treatment with Compound 1. Genes in promoter cluster 1 also show an increase in ARID1A and SMARCA4 binding after Compound 1 treatment, suggesting that inhibition of EZH2 promotes recruitment of the BAF complex to these promoters. Expression analysis of the genes within promoter cluster 1 show that most of these genes are induced by Compound 1, consistent with a dominant PRC2 function to repress these genes and show minimal response to ARID1A re-expression. Of note, while genes in promoter subcluster 1A show similar additive induction of gene expression after Compound 1 treatment and ARID1A re-expression to genes in enhancer-proximal subcluster 1C, these two gene sets only had a small number of genes in common.

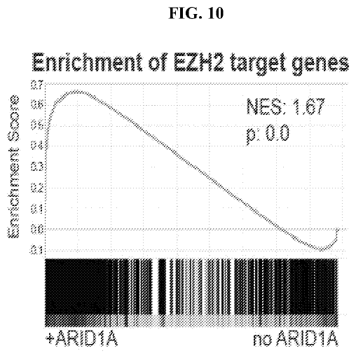

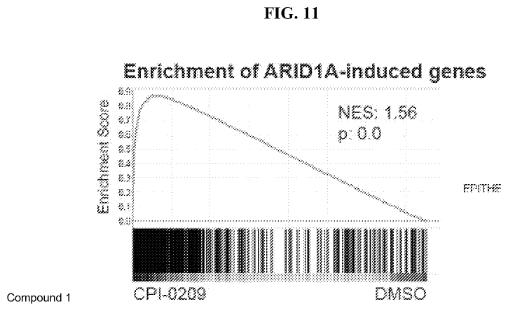

Principal component analysis (PCA) of global gene expression patterns among samples showed that Compound 1 treatment and ARID1A re-expression are the major drivers of gene expression changes. The vast majority (>94%) of genes changed by Compound 1 treatment are significantly upregulated (Log2 fold change (L2FC)≥1), consistent with PRC2's role in gene repression. ARID1A re-expression causes variable changes in gene expression, with ˜62% of significantly altered genes being downregulated, and ˜38% upregulated by ARID1A, consistent with ARID1A's role in both transcriptional repression and activation. GSEA of genes up-regulated by ARID1A re-expression revealed enrichment of EZH2 targets (defined as those induced by Compound 1 treatment and having EZH2 and H3K27me3 peaks). See FIG. 10. Likewise, GSEA of genes upregulated after Compound 1treatment showed enrichment in ARID1A re-expression targets. See FIG. 11. Comprehensive GSEA analysis with the Hallmark Collection indicated that more than half of enriched gene sets are commonly enriched by both treatment with Compound 1 and re-expression of ARID1A, including pathways regulating cell differentiation, immune signaling, and inflammation. Taken together, these data suggest that ARID1A LOF mutations lead to an imbalance of epigenetic gene regulation within a subset BAF- and PRC2-regulated pathways that promote cancer cell growth. These cancer cells depend on PRC2-mediated repression of these gene targets, and Compound 1-mediated EZH2 inhibition may allow for re-expression of these genes to impact cell viability in ARID1A LOF contexts.

### Example 3—Clinical Efficacy

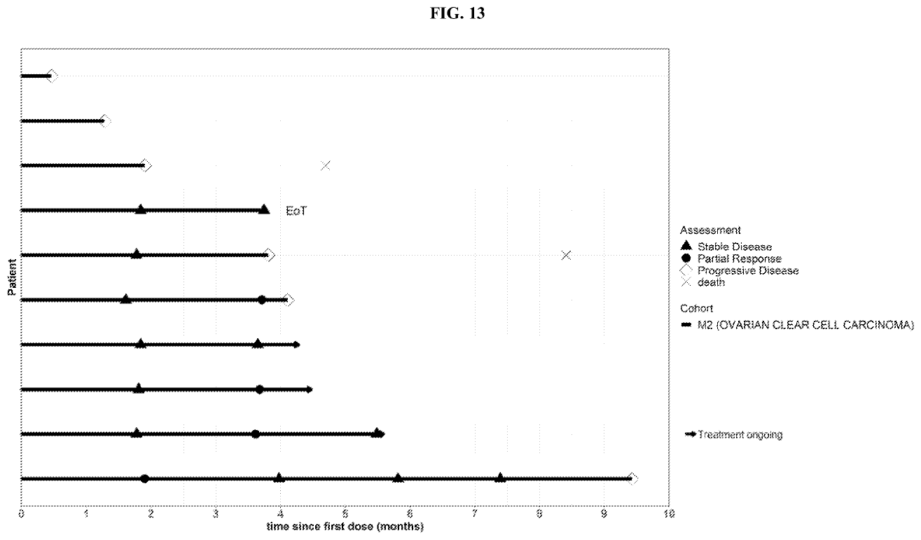

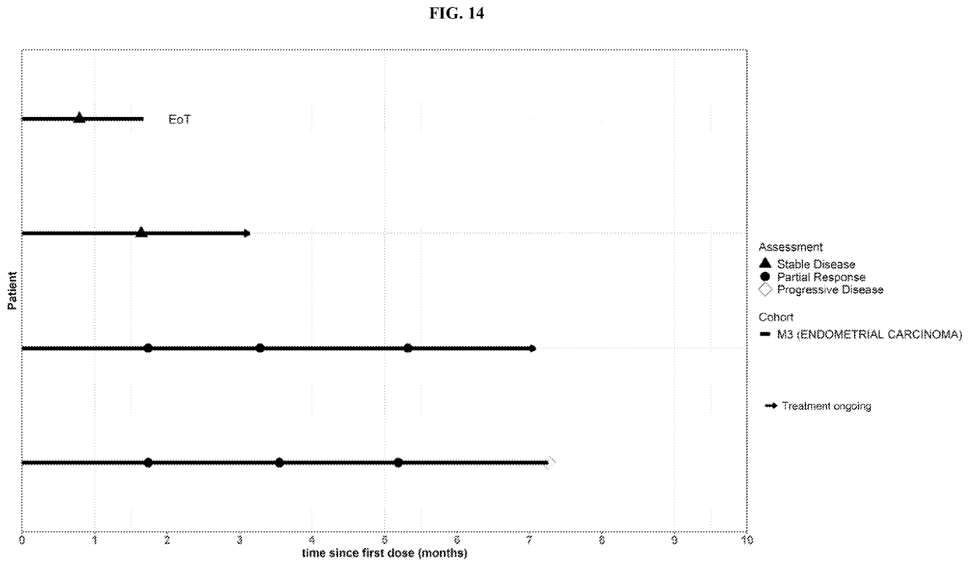

As part of a human clinical phase 2 study (NCT04104776), subjects with ovarian clear cell cancer (Cohort M2) and endometrial carcinoma (Cohort M3) were administered 350 mg/day Compound 1 for the treatment duration shown in FIG. 13 and FIG. 14, respectively. Compound 1 was administered orally as a single agent (monotherapy). The cancers in each subject were known to have ARID1A mutations as determined by next generation sequencing (NGS) prior to treatment. The response by cancer cohort for efficacy evaluable patients at an intermediate cut-off date is shown in Table 3. Assessment of the change in tumor burden is based on RECIST 1.1 criteria. A complete response is characterized as disappearance of all lesions, a partial response is characterized as at least a 30% decrease in the sum of the longest diameter (LD) of target lesions, taking as reference the baseline sum LD; stable disease is characterized as neither sufficient shrinkage to qualify for partial response nor sufficient increase to qualify for progressive disease, taking as reference the smallest sum diameters; and progressive disease is characterized as at least a 20% increase in the sum of the LD of target lesions, taking as reference the smallest sum LD recorded since the treatment started or the appearance of one or more new lesions. To be assigned a status of partial or complete response changes in tumor measurements must be confirmed by repeat assessments no less than 4 weeks after the criteria for response are first met.

TABLE 3

| Category, n (%) | Ovarian (M2) (N = 10) | Endometrial (M3) (N = 4) |
|---|---|---|
| Complete response | 0 | 0 |
| Partial response | 4 | 2 |
| Stable disease | 3 | 2 |
| Progressive disease | 3 | 0 |

In addition, tumor mutational burden was found to be low for most patients with OCCC or EC. TMB-low status was defined as <10 mut/Mb and was assessed by NGS (Tempus xT & Predicine ATLAS targeted panel sequencing).

### Example 4—Comparison of Compound I to other PRC2 Inhibitors in an ARID1A Mutant Bladder Cancer Xenograft Model In Vivo

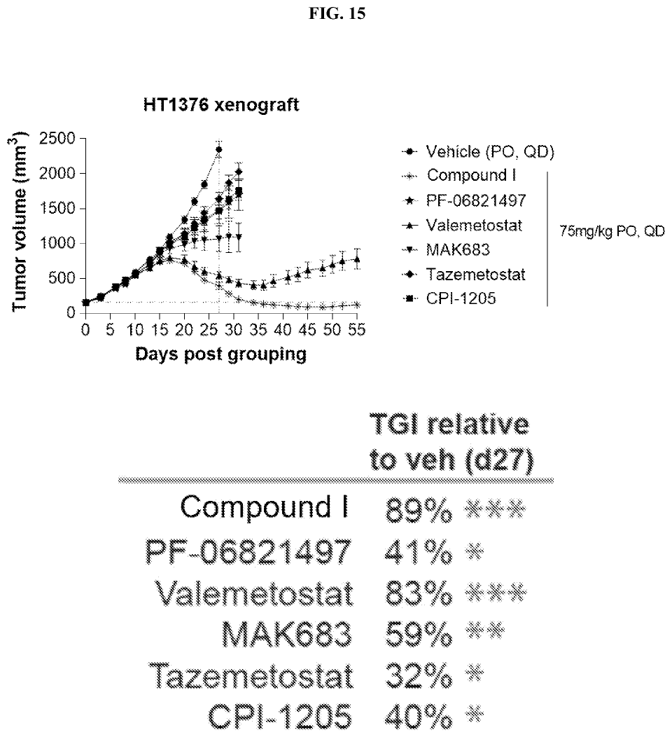

Response of Compound 1 treatment relative to other EZH2, dual EZH1 and EZH2, or EED inhibitors was analyzed in vitro and in vivo. In long-term cell viability assays in vitro Compound 1 had a comparable GI₅₀ to PF-06821497 (˜5 nM) while valemetostat and the EED inhibitor MAK683 were 3-10-fold less potent (GI₅₀: 18 and 73 nM, respectively). First generation EZH2 inhibitors such as tazemetostat (GI₅₀=385 nM) and CPI-1205 (GI₅₀=574 nM) were less potent in viability assays consistent with their lower target affinities and shorter residence times. When administered at the same dose (75 mg/kg QD), Compound 1, valemetostat, and MAK683 showed >50% tumor growth inhibition (TGI) compared to vehicle in an ARID1A mutated HT1376 xenograft mouse model (FIG. 15). At this dose, Compound 1 exhibited significantly more robust and durable anti-tumor activity (p=0.0001) than valemetostat, with tumor growth in the valemetostat arm rebounding after 35 days on treatment, while Compound 1 treated tumors continued to regress below the initial tumor volume by day 37. Tazemetostat, CPI-1205 and PF-06821497 resulted in <50% TGI (32, 40 and 41% TGI at day 27, respectively). Overall, Compound 1 caused complete tumor regression in the ARID1A mutated bladder cancer xenograft model. In mice treated with valemetostat tumor volume decreased over the first 35 days of treatment but did not reach full regression, while mice treated with tazemetostat showed an increase in volume during treatment (FIG. 15). All compounds were well tolerated at 75 mg/kg QD, and plasma exposure of Compound 1 was not greater than any of the other compounds (Table 4).

TABLE 4
Pharmacokinetics properties of EZH2 inhibitors in
the ARID1A mutant HT1376 xenograft mouse model

| | Time | Dose | T_{1/2} | Tₘₐₓ | Cₘₐₓ | AUCₗₐₛₜ |
|---|---|---|---|---|---|---|
| Test Article | point | (mg/kg) | (hr) | (hr) | (ng/mL) | (hg*ng/mL) |
| Compound 1 | Day 1 | 75 | 2.85 | 1.33 | 2550 | 7220 |
| Valemetostat | | | 3.00 | 1.33 | 3090 | 7600 |
| PF-06821497 | | | 1.01 | 0.50 | 3740 | 3380 |
| EEDi | | | 1.18 | 1.00 | 5660 | 13100 |
| CPI-1205 | | | 1.24 | 0.50 | 50800 | 59900 |
| Tazemetostat | | | 1.43 | 0.50 | 9530 | 23200 |

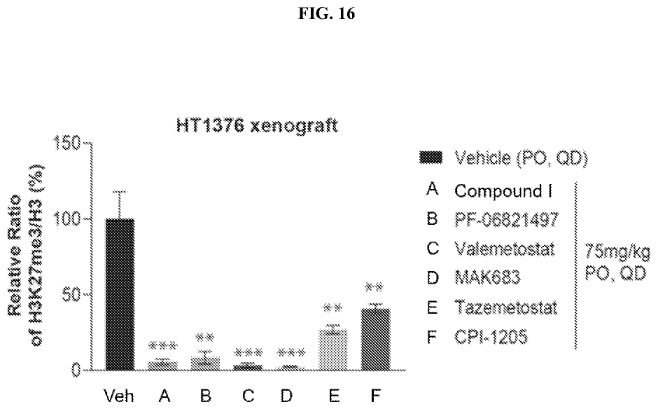

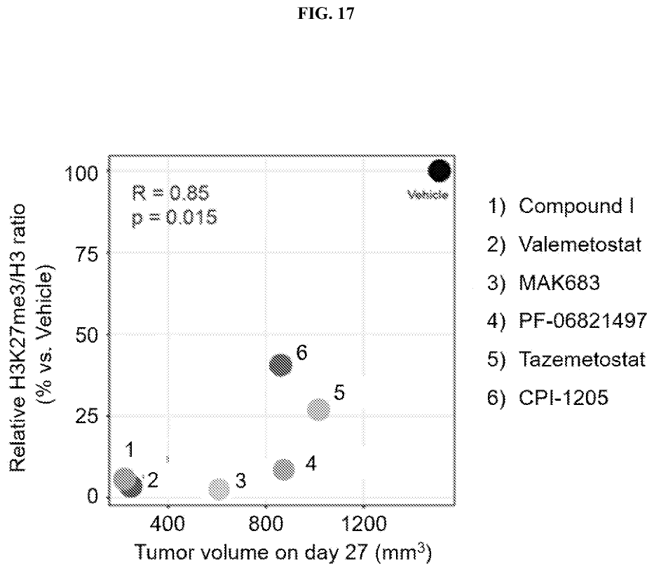

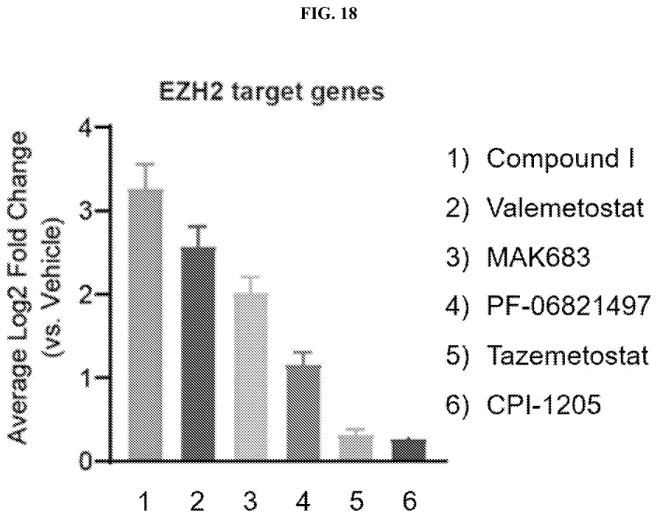

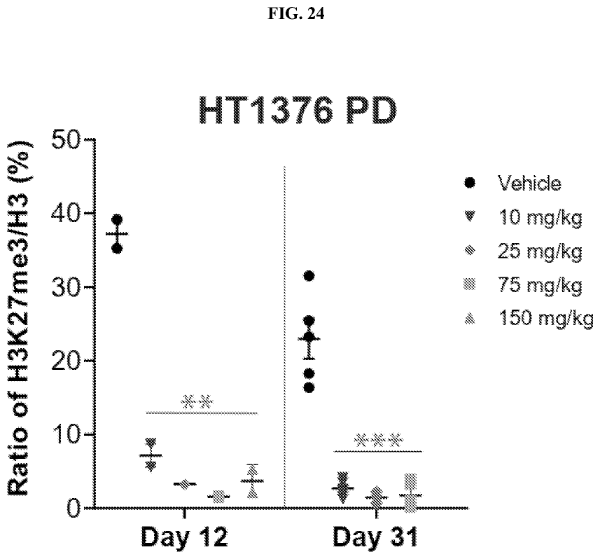

Compounds that resulted in >50% TGI have strong (>90%) reduction in H3K27me3 in tumors collected at day 15, while those that had weaker TGI retained higher levels of H3K27me3 (FIG. 16), demonstrating a link between tumor response and degree of H3K27me3 reduction (FIG. 17). However, the magnitude of reduction in global H3K27me3 level changes >90% could not distinguish differences in TGI of the most efficacious compounds, Compound 1, valemetostat and MAK683. Gene expression profiling from tumors at day 15 revealed that Compound 1 induces significantly more EZH2/PRC2 target genes (Table 5) than any other compound, and that overall changes in gene expression level are consistent with reduced H3K27me3 and tumor response (FIG. 18 and FIG. 24). Contrary to global changes in H3K27me3 levels, greater numbers of altered genes correlated with greater magnitude in TGI, suggesting that gene expression changes may be a preferred biomarker to relate target inhibition and efficacy.

TABLE 5
Gene sets significantly enriched (FDR < 0.01) from the
C2 curated gene set list from MSigDB in the enhancer subclusters.

| | |
|---|---|
| | FDR q- |
| Gene Set | value |

Gene sets enriched in enhancer-proximal subcluster 1C

| | |
|---|---|
| GRYDER_PAX3FOXO1_ENHANCERS_IN_TADS | 2.50E−09 |
| PEREZ_TP53_TARGETS | 3.54E−09 |
| BENPORATH_ES_WITH_H3K27ME3 | 1.70E−08 |
| PUJANA_BRCA1_PCC_NETWORK | 3.28E−08 |
| MEISSNER_BRAIN_HCP_WITH_H3K4ME3_AND_H3K27ME3 | 3.77E−08 |
| BHAT_ESR1_TARGETS_VIA_AKT1_UP | 6.60E−08 |
| MILI_PSEUDOPODIA_HAPTOTAXIS_DN | 2.54E−07 |
| TIEN_INTESTINE_PROBIOTICS_24 HR_UP | 6.61E−07 |
| REACTOME_METABOLISM_OF_LIPIDS | 9.75E−07 |
| NUYTTEN_EZH2_TARGETS_DN | 1.21E−06 |

Gene sets enriched in enhancer-proximal subcluster 1B

| | |
|---|---|
| MIKKELSEN_MCV6_HCP_WITH_H3K27ME3 | 5.75E−16 |
| BENPORATH_ES_WITH_H3K27ME3 | 6.72E−16 |
| MIKKELSEN_MEF_HCP_WITH_H3K27ME3 | 3.34E−12 |
| BENPORATH_EED_TARGETS | 7.01E−12 |
| MEISSNER_BRAIN_HCP_WITH_H3K4ME3_AND_H3K27ME3 | 8.34E−12 |
| BENPORATH_SUZ12_TARGETS | 5.62E−10 |
| MARTENS_TRETINOIN_RESPONSE_UP | 1.12E−09 |
| BENPORATH_PRC2_TARGETS | 5.05E−09 |
| MEISSNER_NPC_HCP_WITH_H3K4ME2_AND_H3K27ME3 | 2.14E−07 |
| PEREZ_TP53_TARGETS | 3.10E−07 |
| MEISSNER_BRAIN_HCP_WITH_H3K27ME3 | 4.74E−06 |
| REACTOME_MUSCLE_CONTRACTION | 1.19E−05 |
| SANSOM_APC_TARGETS_DN | 1.22E−05 |
| REACTOME_CARDIAC_CONDUCTION | 2.69E−05 |
| ROZANOV_MMP14_TARGETS_UP | 2.69E−05 |
| MIKKELSEN_NPC_HCP_WITH_H3K27ME3 | 5.49E−05 |
| LIM_MAMMARY_STEM_CELL_UP | 6.14E−05 |
| NABA_MATRISOME | 6.14E−05 |
| MEISSNER_NPC_HCP_WITH_H3K4ME2 | 7.56E−05 |
| BLANCO_MELO_BRONCHIAL_EPITHELIAL_CELLS_INFLUENZA_A_DEL_NS1_INFECTION_UP | 8.74E−05 |

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating a cancer in a subject comprising administering to the subject an effective amount of a compound having the formula: 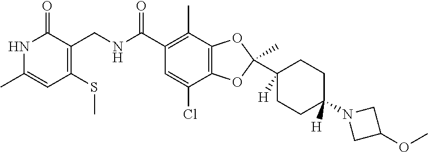
or a pharmaceutically acceptable salt thereof, wherein the cancer has at least one ARID1A mutation.

2. The method of claim 1, wherein the cancer is selected from bladder, breast, endometrial, gastric, colon, colorectal, pancreatic, cholangio, stomach, hepatocellular, liver, lung, melanoma, and ovarian cancer.

3. The method of claim 1 or 2, wherein the cancer is selected from bladder cancer, endometrial cancer, and ovarian clear cell carcinoma.

4. The method of any one of claims 1 to 3, wherein the cancer is bladder cancer.

5. The method of claim 4, wherein the bladder cancer is urothelial carcinoma.

6. The method of claim 4 or 5, wherein the bladder cancer is advanced urothelial carcinoma.

7. The method of any one of claims 1 to 3, wherein the cancer is endometrial cancer.

8. The method of any one of claims 1 to 3, wherein the cancer is ovarian cancer.

9. The method of claim 8, wherein the cancer is ovarian clear cell carcinoma.

10. The method of any one of claims 1 to 9, wherein the at least one ARID1A mutation is a loss of function (LOF) mutation

11. The method of any one of claims 1 to 10, wherein the compound or pharmaceutically acceptable salt is administered for a period of at least 6 days.

12. The method of any one of claims 1 to 11, wherein administration restores ARID1A expression.

13. The method of any one of claims 1 to 12, wherein administration increases SMARCA4 binding.

14. The method of any one of claims 1 to 13, wherein administration increases ARID1A binding.

15. The method of any one of claims 1 to 14, wherein administration reduces global H3K27me3 levels.

16. The method of any one of claims 1 to 15, wherein administration results in durable reduction of tumor volume.

17. The method of any one of claims 1 to 16, wherein administration drives re-expression of repressed genes.

18. The method of any one of claims 1 to 17, wherein the cancer is characterized by a low tumor mutational burden.
